(19) United States
(12) Patent Application Publication
(10) Pub. No.: US 2026/0213007 A1
(43) Pub. Date: Jul. 23, 2026

(54) TRACKING SYSTEM AND METHOD FOR PATIENT TRANSFER DURING IMAGING AND TREATMENT PROCEDURES

(71) Applicants: QFIX SYSTEMS, LLC, Avondale, PA (US); OTTO-VON-GUERICKE UNIVERSITÄT MAGDEBURG, Magdeburg (DE)

(72) Inventors: Robert Kowal, Magdeburg (DE); Bennet Lamers, Magdeburg (DE); Remigiusz Kwapik, Bremerhaven (DE); Ivan Fomin, Magdeburg (DE); Oliver Thieme, Weißenfels (DE); Enrico Pannicke, Magdeburg (DE); Thomas Gerlach, Magdeburg (DE); Daniel Coppens, Avondale, PA (US)

(21) Appl. No.: 19/137,843

(22) PCT Filed: Dec. 7, 2023
(86) PCT No.: PCT/US2023/082877

§ 371 Date: Jun. 11, 2025

Related U.S. Application Data
(60) Provisional application No. 63/432,861, filed on Dec. 15, 2022.

(51) Int. Cl. G16H 40/63; A61B 5/00; A61G 7/10; G01B 11/14; G06T 7/246; G06T 7/73; H04N 23/56; H04N 23/67

(52) U.S. Cl. CPC G16H 40/63; A61B 5/704; A61G 7/1021; G01B 11/14; G06T 7/248; G06T 7/74; H04N 23/56; H04N 23/671; G06T 2207/30004; G06T 2207/30204

(57) ABSTRACT

A system supports a patient and tracks the position and/or orientation of a patient support. The system has a patient transfer apparatus; at least one reference surface; a sensor acquiring input related to a position of the patient transfer apparatus; at least one processor to process and convert the input from the at least one sensor; at least one transmitter to transmit the input from the at least one sensor to the at least one processor; and a tracking feature disposed on the reference surface. The sensor is coupled to the patient transfer apparatus to detect the tracking feature disposed in or on the at least one reference surface and the at least one transmitter transmits the input from the sensor to the at least one processor for processing and conversion into coordinates within a reference frame in at least one degree of freedom.

### CROSS-REFERENCE TO RELATED APPLICATION

This application claims priority to U.S. Provisional Application No. 63/432,861, filed Dec. 15, 2022, and the contents of which is incorporated herein by reference in its entirety for all purposes.

### FIELD OF THE INVENTION

The present invention is directed to system and methods for tracking the coordinates of a patient transfer apparatus with selected sensors, transmitters, processors, and reference surfaces having tracking features capable of being detected by the sensors.

### BACKGROUND OF THE INVENTION

An objective problem exists of how to track the position and/or orientation of a patient transfer apparatus or support while it is in use on diagnostic and therapeutic modalities, independent of the inbuilt or external tracking measures of the modality, which is especially needed in light of today's modern multimodal diagnostic and treatment procedures.

### SUMMARY OF THE INVENTION

Generally, the present invention is directed to system and methods for tracking the coordinates of a patient transfer apparatus with selected sensors, transmitters, processors, and reference surfaces having tracking features capable of being detected by the sensors.

A system configured to support a patient and track the coordinates of a patient support within a reference frame is provided. The system comprises a patient transfer apparatus configured to support and transfer a body of the patient; at least one reference surface; at least one sensor capable of acquiring input related to the position of the patient transfer apparatus; at least one processor, configured to process and convert the input from the at least one sensor; at least one transmitter, configured to transmit the input from the at least one sensor to the at least one processor; and at least one tracking feature disposed on the reference surface, having at least one encoding index adapted to be detected by the at least one sensor, and further representing a unique position within the reference surface. The at least one sensor is coupled to the patient transfer apparatus, such that the at least one sensor is positioned to detect the at least one tracking feature disposed in a reference surface. The at least one sensor is also connected to the at least one transmitter. The at least one transmitter is connected to the at least one processor such that the at least one transmitter is capable of transmitting the input from the at least one sensor to the at least one processor for processing and conversion into a reference frame in at least one degree of freedom.

A method of use of the system as described is also provided. The method comprises the steps of: a) positioning a patient transfer apparatus on at least one of a support surface or an imaging or treatment modality, wherein at least one of the support surface or imaging or treatment modality further has at least one reference surface; b) positioning a patient on the patient transfer apparatus; c) acquiring the initial coordinates of the patient transfer apparatus within a reference frame; d) moving the patient transfer apparatus; and e) acquiring the new coordinates of the patient transfer apparatus.

A method of determining the coordinates of a patient transfer apparatus within a reference frame is additionally provided. This method comprises the following steps: a) positioning an at least one sensor coupled to patient transfer apparatus, such that the at least one sensor is capable of detecting at least one tracking feature positioned within a reference surface; b) activating the at least one sensor, such that it is capable of acquiring input, the input including at least one tracking feature disposed in the reference surface and the at least one tracking feature represents a unique position defined in the reference surface; c) acquiring input, using the at least one sensor, and the input includes at least one tracking feature disposed in the reference surface, the at least one tracking feature representing a unique position within the reference surface; d) transmitting the input, using the at least one transmitter, from the at least one sensor to at least one processor; and interpreting the input, using the at least one processor, from the at least one sensor, including translating selected structures of the at least one tracking feature into a coordinates within the reference frame.

### BRIEF DESCRIPTION OF THE DRAWINGS

FIG. 1 shows a system according to an embodiment of the present invention.

FIG. 2 shows a system according to an embodiment of the present invention including an air source and air bearing.

FIG. 3 shows a system according to an embodiment of the present invention including a support surface.

FIG. 4 shows an embodiment of the at least one reference surface of the present invention.

FIG. 5 shows yet another embodiment of the at least one reference surface of the present invention.

FIG. 6 shows yet another embodiment of the at least one reference surface of the

FIG. 7 shows yet another embodiment of the at least one reference surface of the present invention.

FIGS. 8A-D show selected positions of the at least one or plural sensor coupled to the patient transfer apparatus.

FIG. 9 shows the position of the at least one or plural sensors during a rotation of the patient transfer apparatus of the system according to an embodiment of the present invention while it is positioned on an at least one reference surface.

FIG. 10 shows 6 degrees of freedom relative to the patient transfer apparatus according to an embodiment of the present invention.

FIG. 11 shows an alternate embodiment of the system of the present invention wherein the at least one processor is coupled to the at least one patient transfer apparatus.

FIG. 12 shows an embodiment of the system of the present invention having supplementary tracking means.

FIG. 13 shows an embodiment of the system of the present invention wherein the at least one processor is connected to another unit such as a display.

FIG. 14 shows an embodiment of the system of the present invention wherein plural sensors are connected to a single at least one transmitter.

FIG. 15 shows an embodiment of the system of the present invention.

FIG. 16 shows yet another embodiment of the present invention

FIG. 17 shows a block diagram of selected elements of an embodiment of the system of the present invention.

FIG. 18 shows selected examples of the at least one tracking feature and the at least one encoding index according to an embodiment of the present invention.

FIG. 19 shows an exemplary method of using the system of the present invention.

FIG. 20 shows an exemplary method of determining the coordinates of a patient transfer apparatus within a reference frame.

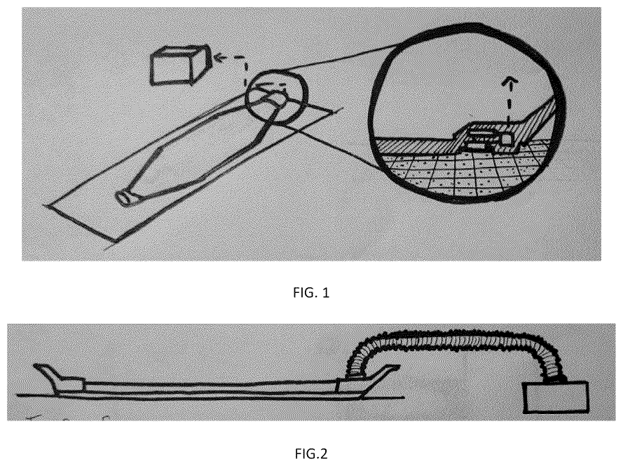

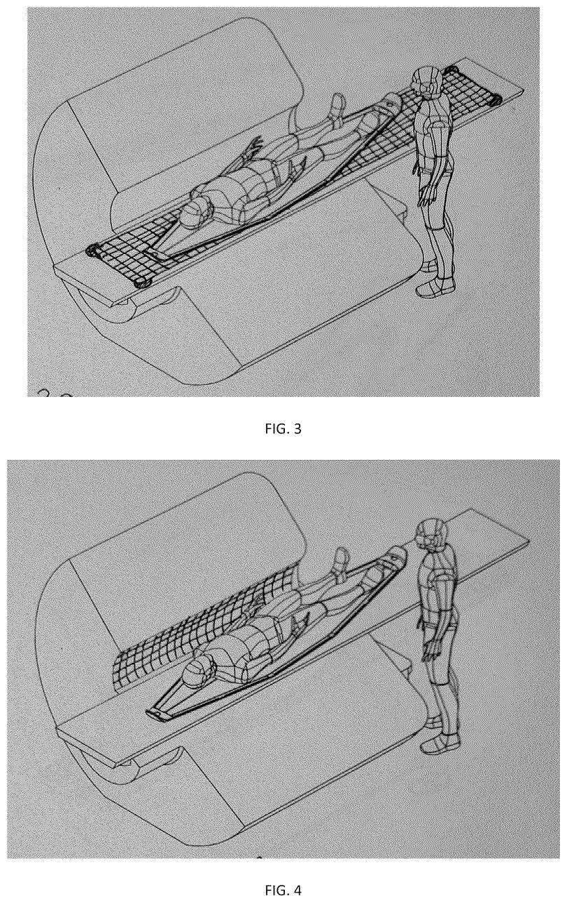

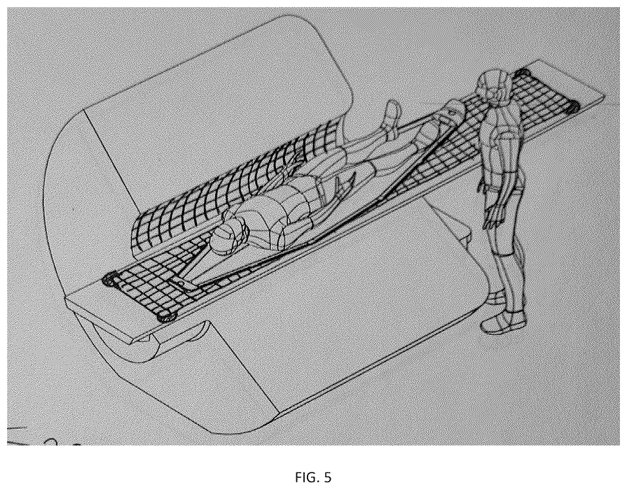

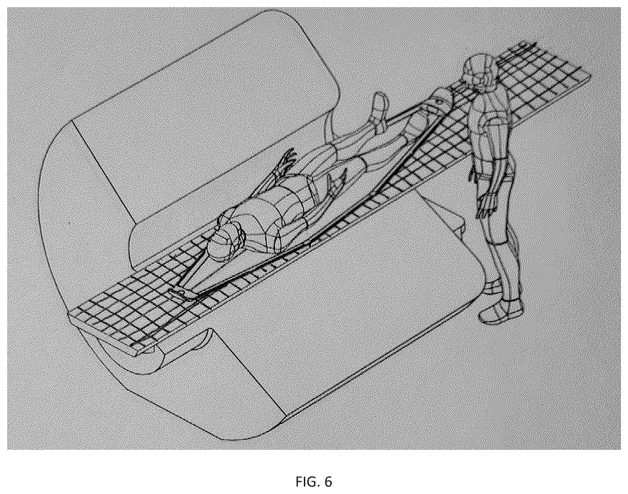

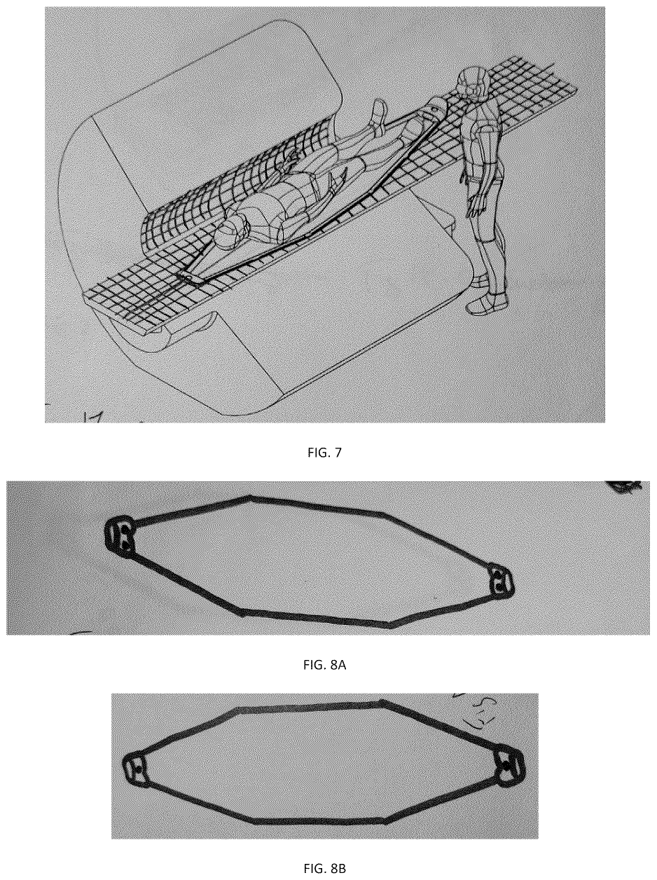

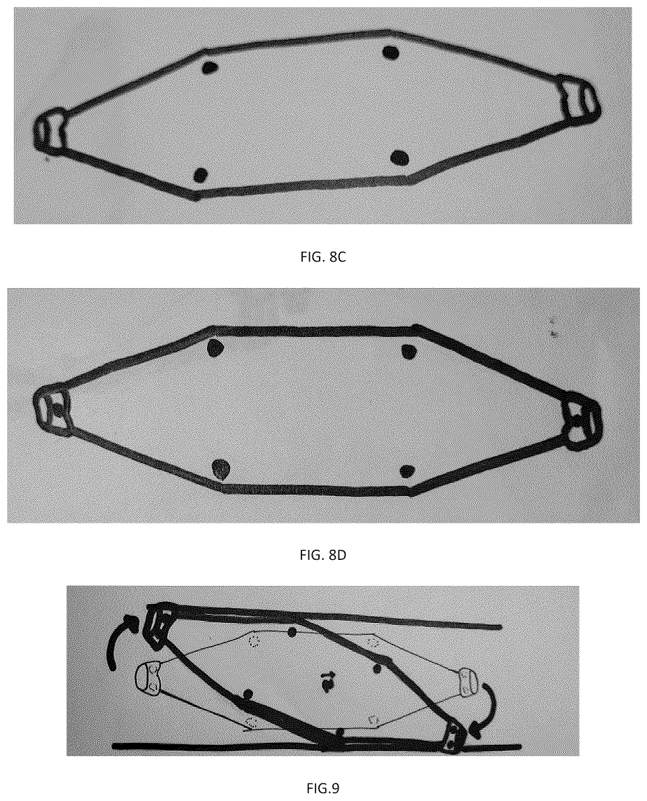

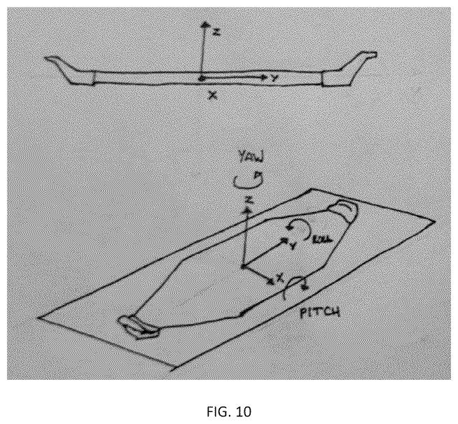

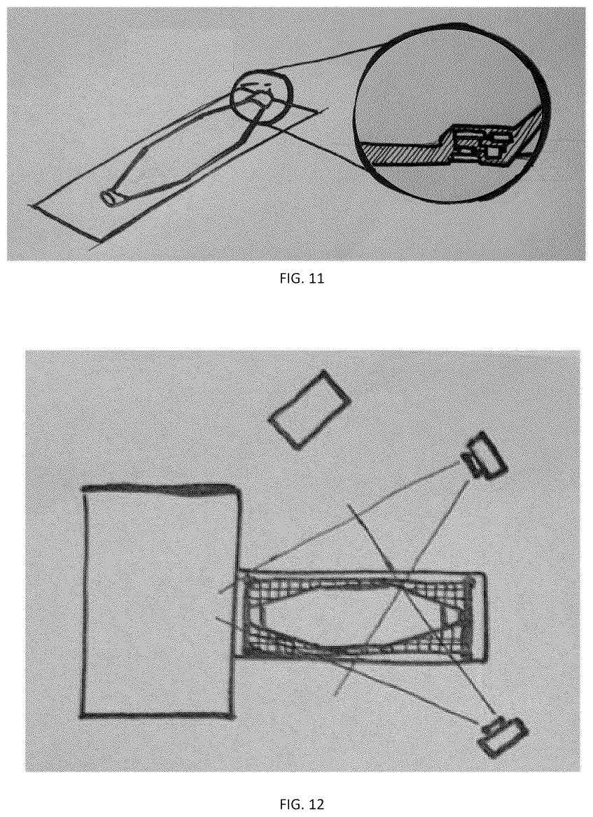

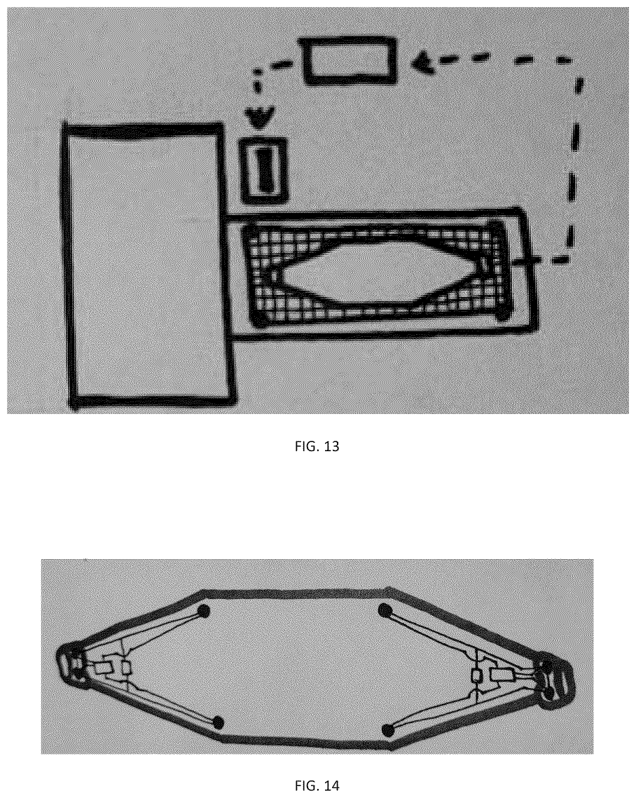

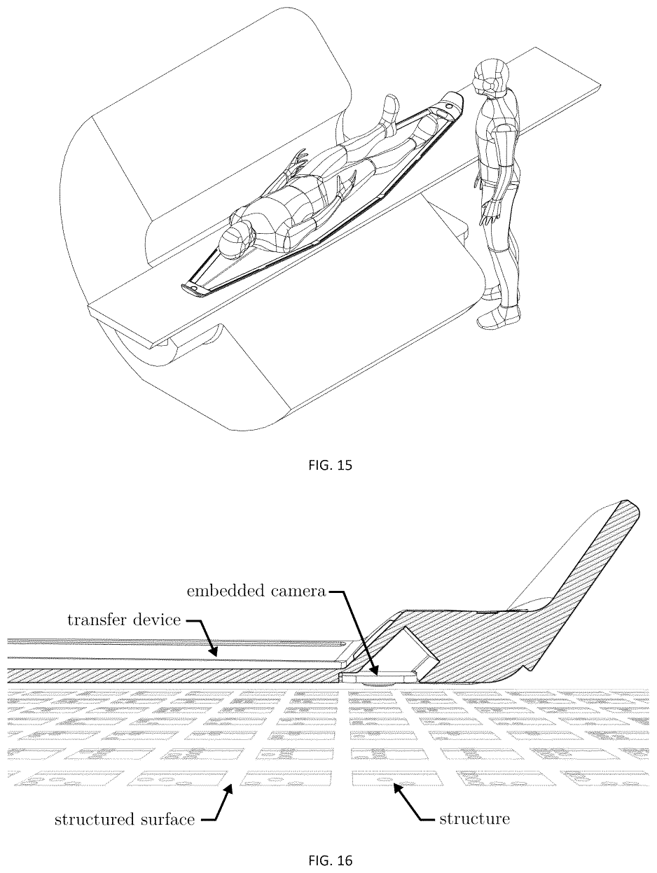

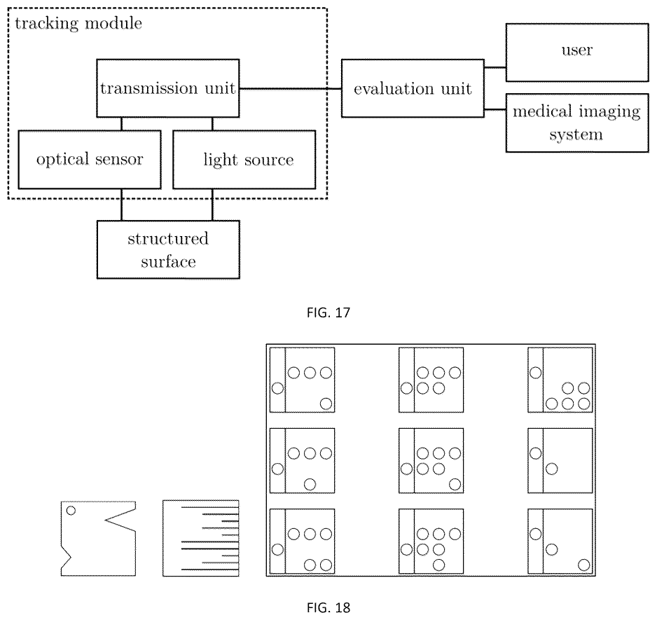

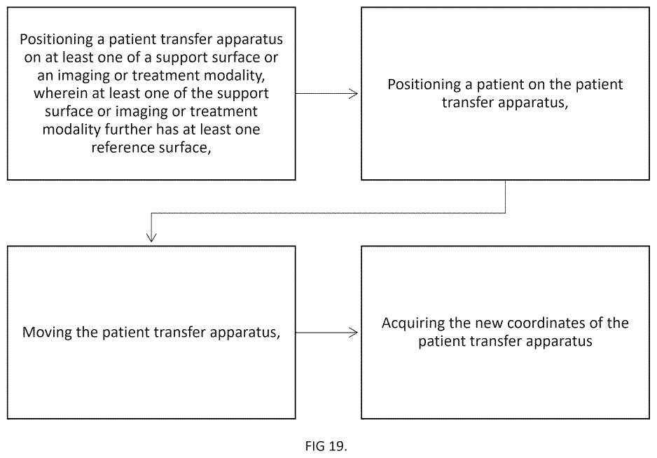

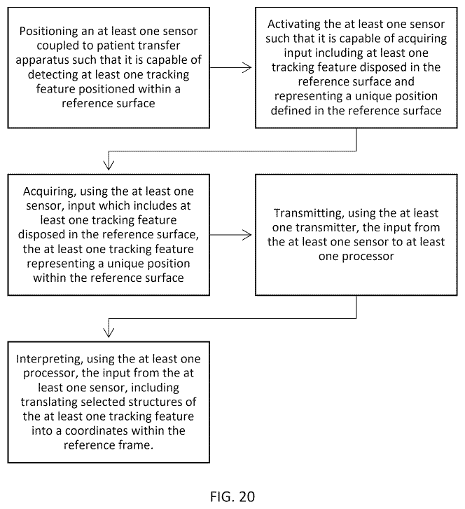

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

In typical practice today, clinicians in the fields of interventional radiology, radiation therapy, neurology, cardiology, and other spaces often utilize multiple techniques to prepare, image, and treat patients for complex, image-guided procedures. Such procedures may utilize one of x-ray imaging, computed tomography (CT), magnetic resonance imaging (MRI), fluoroscopy, angiography, among others, or combinations thereof to combine the strengths of each modality while minimizing undesirable tradeoffs. As treatment and imaging modalities are further developed and combined for optimal patient outcomes, clinicians are turning to solutions for enhancing workflow. Often this is across these multiple modalities and there is a desire to improve throughput speed in setup and treatment and increase valuable machine uptime. Patient transfer systems provide such a solution. They can be used to support, position, transfer, and transport patients among various modalities and across a variety of tables, beds, and support surfaces. Exemplary systems having a movable patient transfer device or support drastically reduce time and personnel required for transferring and positioning the patient. Additionally, such systems are often usable independent of the selected modality and enable free movement outside of the constraints of the selected modality's support surfaces, including free movement and rotation within the bore. However, this free movement introduces a new complication. It is ideal that position and orientation of the region of interest is known or tracked during the procedure, and it is especially important that tracking does not interfere with the workflow of the clinical team while still providing robust information, particularly during image-guided patient procedures. In particular, safety features of the imaging or treatment modality may rely on tracking the patient position. Typical modalities often provide such tracking or indexing through the fixed patient couches or supports of the imaging systems. For example, exemplary integrated patient supports may have electromechanically guided drives which have their positions controlled and detected by the scanner itself. Alternately, such exemplary modalities may incorporate reference points and laser markings projected onto the patient. Such methods do not allow recording the position and orientation of a freely movable patient transfer apparatus. Additionally, other solutions, such as position tracking systems located outside the scanner, or inside the scanner bore above the transfer apparatus, require an unobstructed line of sight to the object to be tracked. This may not be achievable, especially in typical imaging modalities, given the tight space constraints within the bore of such modalities and the presence of clinicians working in the region of interest, positioning of other critical therapeutic and support equipment, and the use of surgical drapes and other objects. All of these may obstruct the line of sight of such systems, rendering them unable to reliably provide a position and orientation information. Thus, there is an objective problem of how to track the position and/or orientation of a patient transfer apparatus or support while it is in use on a variety of diagnostic and therapeutic modalities, independent of the inbuilt or external tracking measures of the modality. The present invention provides a solution to this.

Generally, the present invention is directed to a system and methods for use of a system configured to track the coordinates of a patient transfer apparatus with selected sensors, transmitters, processors, and reference surfaces having tracking features capable of being detected by the sensors.

According to a first aspect of the invention, a system configured to support a patient and track the coordinates (and thus the position and/or orientation) of a patient support is provided. The system of the present invention comprises a patient transfer apparatus configured to support and transfer a body of a patient. The patient transfer apparatus is generally configured to be suitable for use with the different imaging, treatment, transport modalities, or combinations thereof within a clinical workflow. For example, in a preferred embodiment, the patient transfer apparatus comprises materials, such that at least a portion of the patient transfer apparatus disposed in a region of interest is x-ray homogeneous and radiolucent and further is capable of being used in magnetic resonance imaging environments. In a further embodiment, the patient transfer apparatus comprises non-porous materials cleanable with typical cleaning procedures known to hospital staff and clinician, such that fluids from the procedure do not incidentally contaminate the device. The patient transfer apparatus is further configured such that it is movable relative to an underlying support surface. In a preferred embodiment, the patient transfer apparatus is further configured such that is movable independently relative to an underlying support surface. Non-limiting examples of the underlying support surface may include a trolley, bed, stretcher, backboard, a couch top or table, or other supporting surface capable of receiving the patient transfer apparatus. In yet another embodiment, the support surface may be connected to or is an integral part of an imaging or treatment modality, such as an MR or CT scanner table. Alternately, the support surface may be a separate overlay installable on supporting structures known to one skilled in the art, such as on any one of the preceding exemplary patient supports. The patient transfer apparatus may be provided with additional features which enable such movement. For example, measures to reduce friction between the underside of the patient transfer apparatus and the support surface may be employed. These friction-reducing measures may include, but are not limited to, low friction skates or pads, rollers, air bearings, or other means known to one having ordinary skill in the art. In a preferred embodiment, the patient transfer apparatus is provided with at least one air bearing located on an underside of the patient transfer apparatus. In this embodiment, the system is additionally provided with at least one air source. The at least one air source is adapted to deliver air to the at least one air bearing when activated, thereby creating airflow through the at least one air bearing. The airflow thus creates a low-friction region underneath the patient transfer apparatus, enabling reduced effort required to move the patient transfer apparatus. Preferably, the patient transfer apparatus is capable of being moved in at least one degree of freedom. In a preferred embodiment, this at least one degree of freedom is at least one of lateral or longitudinal translation. In a further preferred embodiment, the patient transfer apparatus is further shaped such that it is capable of being rotated about an axis extending perpendicularly from a top side of the patient transfer apparatus and is thus capable of being moved in at least three degrees of freedom (e.g., both lateral and longitudinal translation, as well as yaw rotation). In yet another embodiment, the patient transfer apparatus is capable of being moved in at least six degrees of freedom, including height translation, rotation about a longitudinal axis of a patient transfer device (e.g., roll), and rotation about a lateral or transverse axis (pitch). In yet another embodiment, additional degrees of freedom may be enabled through the use of accessory support devices such as stirrups which have ranges of motion relative to the patient transfer apparatus. These embodiments featuring enhanced ability to move about in multiple degrees of freedom are beneficial to improve positioning of the patient for procedures and enhance clinician access to the region of interest.

The system is capable of tracking and determining the coordinates of the patient transfer apparatus within a reference frame to indicate the position and/or orientation of the patient transfer apparatus as the patient transfer apparatus moves according to various degrees of freedom. The system further comprises at least one reference surface which defines the reference frame in at least one degree of freedom. In a preferred embodiment, the reference surface is incorporated into at least one of the support surface and the structure of the imaging or treatment modality. The at least one reference surface may be a substantially flat plane or may be a curved surface. In a preferred embodiment, the at least one reference surface is defined by the support surface. In a further embodiment the at least one reference surface is defined by a support surface which is integrated into an exemplary imaging or treatment modality. In an alternate embodiment, the at least one reference surface is defined by a support surface which is an overlay configured to be positioned or indexed atop a table or patient support of an imaging or treatment modality. In yet another embodiment, the at least one reference surface is positioned on the interior of the cylindrical bore of an exemplary imaging machine. In a further embodiment, multiple reference surfaces may be positioned adjacent to one another such that they form a series of reference frames and coordinate system in multiple degrees of freedom. In yet another embodiment, the multiple reference surfaces may represent and be positioned adjacent multiple different regions of the patient's anatomy where position and/orientation tracking is considered critical. An exemplary embodiment may include an overlay containing a first reference surface, which may be located atop the patient support of an imaging modality, as well as a second reference surface positioned on the interior of the bore of the imaging modality. In this embodiment, a coordinate system for at least four degrees of freedom is provided, wherein the first reference surface provides at least coordinates for longitudinal and lateral translation and yaw of the patient transfer apparatus, and the second reference surface provides at least coordinates to determine roll of the patient transfer apparatus. The system further comprises at least one tracking feature, which is disposed in the reference surface. The at least one tracking feature further has at least one encoding index, which represents a unique position within the reference surface and unique coordinates within the reference frame. In a preferred embodiment, plural tracking features are disposed in the at least one reference surface, with each individual encoding index representing a unique coordinate within the reference frame. The plural tracking features may be discrete or continuous. Additionally or optionally,, the at least one encoding index a tracking feature differs from another of the least one encoding index of another tracking feature, and further differ across the spatial area of the at least one reference surface in a predictable manner, thereby facilitating interpretation by way of computer vision or other sensing and processing means.

To detect the at least one tracking feature within the at least one reference surface, the system is provided with at least one sensor capable of acquiring input related to the position of the patient transfer apparatus. More specifically, the at least one sensor may be adapted to detect the at least one encoding index of the at least one tracking feature disposed in the at least one reference surface. The at least one sensor may additionally be adapted to detect other characteristics of the orientation or movement of the patient transfer apparatus not directly obtainable from the at least one tracking feature of the at least one reference surface. Preferably, the at least one sensor is coupled to the patient transfer apparatus such that it is positioned to detect the at least one tracking feature disposed in the at least one reference surface. The at least one sensor may comprise cameras, charge coupled devices, infrared sensors, laser sensors (including LIDAR sensors, laser Time of Flight (ToF), and laser phase shift sensors), accelerometers, gyroscopes, haptic sensors, and other sensors known to one having ordinary skill in the art. The selection of the appropriate sensor and its positioning relative to the patient transfer apparatus should be made with an understanding of environmental and clinical constraints. For example, magnetic resonance imaging environments may interfere or inhibit the functioning of sensors which rely on certain types of magnetism or electrical functioning, especially in the strongest parts of the bore of the MRI scanner, so preference may be given to sensors which can maintain their function within an MR environment. Non-limiting examples of such sensors include BNO08x sensors by Adafruit. In another example, the positioning of the at least one sensor close to the region of interest may create artifacts in diagnostic imaging modalities, or, specific to x-ray imaging and treatment modalities, may create an undesirable region of inhomogeneity. In a further example, the geometric configuration of an underlying patient table may mean that a sensor may be outside of the bounds of the patient table when extremely rotated in certain degrees of freedom, and thus potentially unable to sense. Additionally, how the at least one encoding index and the at least one tracking feature are specifically disposed in the at least one reference surface may differ depending on the type of sensor or sensors selected and the level of sensitivity or accuracy required for the desired application. For example, discrete shapes etched or printed into the at least one reference surface are suitable for use with visual optical sensors such as cameras and charge coupled devices, whereas haptic sensors and LiDAR and other laser sensors may be adapted to detect changes in depth or surface reflectivity of etched or carved symbols, lines, or other indicators disposed in at least one reference surface. In certain embodiments, the printed or etched features may not be visible to the human eye. For example, the at least one encoding index and/or the at least one tracking feature may be printed in ink adapted to be detected by sensors which read wavelengths outside the visible spectrum of light, such as ultraviolet or infrared.

To adapt for these situations and to potentially derive the benefits of the various types of sensors, in preferred embodiments, plural sensors are provided and positioned in a manner such that at least one of the plural sensors can receive input. Additionally, plural sensors can be configured such that they (i) provide additional points of data to better localize the coordinates of the patient transfer apparatus within the reference frame, (ii) provide additional coordinate data to calculate derived information regarding orientation such as rotation angles, and/or to (iii) provide redundancy if a sensor fails or is otherwise unable to obtain a reading. In a further embodiment, the plural sensors may be the same type of sensors or may be different types of sensors. Plural sensors of different types may be adapted to acquire different inputs related to the coordinate position of the patient transfer apparatus within the reference frame. For example, a certain sensor may be configured to obtain a reading related to one degree of freedom, such as translational lateral and longitudinal location, while another type of sensor may be configured to obtain a different reading related to a different degree of freedom, such as vertical displacement. Alternately, plural sensors may be provided, with one sensor primarily configured to detect the at least one encoding index of the at least one tracking feature, and another sensor configured to support the detection of the at least one encoding index of another tracking feature. In yet another alternate embodiment, plural sensors may be provided, with one sensor configured to detect the at least one encoding index of the at least one tracking feature, and another sensor configured to provide additional information related to the position or orientation of the patient transfer apparatus not obtainable or derivable from the information provided by the first sensor. Input acquisition from the at least one sensor or plural sensors may be (i) continuous or (ii) triggered by user input and/or by other triggers, or (iii) combinations thereof. Such triggers may include, by way of non-limiting example, deactivation of an air source, cessation of movement for a period of time, or a signal received from an imaging or treatment modality or another piece of equipment within the area. This can vary across the various plural sensors disposed in the patient transfer apparatus, with some sensors “always on” and others only acquiring an input, for example, when the device is not moving.

The following paragraphs describe selected non-limiting embodiments of combinations of sensors and tracking features and their respective orientations. These are provided solely as illustrative examples. It is understood that other combinations or arrangements of these features may be conceivable to one having ordinary skill in the art to achieve a resulting system as described and claimed.

In an embodiment, the plural sensors include plural cameras coupled to an underside of the patient transfer apparatus. In this embodiment the at least one reference surface is positioned beneath the patient transfer apparatus, most preferably on a support surface. The support surface includes plural tracking features which contain respective encoding indexes adapted to be detected by the plural cameras. In a particular embodiment, the at least one encoding index of the at least one tracking feature contains binary-coded information implemented by the presence or absence of geometric markers at predefined positions within each structure. These geometric markers may include circles, boxes, points, crosses, or other markers known to one having ordinary skill in the art. Alternative versions of the at least one tracking feature featuring analog coding can also be implemented by calculating information about lengths and widths of specific geometric features, acting as the at least one encoding index, from the image data. The orientation of straight lines or boundaries in the acquired image, in relation to a reference, can additionally be used to calculate the orientation of the patient transfer apparatus. In a preferred embodiment, the encoding indexes of the plural tracking features can be used to derive the coordinates within the reference frame in at least 3 degrees of freedom. To better visualize the plural tracking features, a means of illuminating the field of view of the cameras may be provided, such as one or more lights surrounding the cameras'lens. These plural cameras can be arranged in multiple ways. In an embodiment, one or more of the plural cameras may be disposed at each of a superior end and an inferior end of the patient transfer apparatus. Each of the plural cameras provide location input for their respective end. In a further embodiment, multiple cameras are provided at each of the superior and inferior end of the patient transfer apparatus, thereby enabling more precise triangulation of both longitudinal and lateral translational location as well as improved coordinate capture for the derivation of yaw. Additionally, multiple cameras located at each superior and inferior ends of the patient transfer apparatus provide redundancy if one camera malfunctions or is otherwise unable to obtain an input (for example, when the patient transfer apparatus is rotated and at least one end is partially disposed beyond the boundary of the support surface).

Alternately or in addition to the previous embodiment, plural cameras may be disposed within an intermediate region of the patient transfer apparatus located between the respective superior and inferior ends of the patient transfer apparatus and are spaced apart. This provides additional location information and can ensure that fewer cameras are unavailable due to rotation of the patient transfer apparatus beyond the boundary of the support surface.

Alternately or in addition to the previous embodiment, the plural sensors may include at least one laser sensor. The at least one laser sensor is preferably capable of acquiring corresponding changes in depth or reflectivity within a surface. In such an embodiment, the at least one encoding index of the at least one tracking feature may be represented by etched features with varying depth, similar to the pits and lands of a compact disc, or the presence or absence of mirrors or lenses such as Fresnel lenses arranged in such a way that the change in reflectivity is capable of being detected by the at least one sensor. In a further embodiment wherein the at least one laser is provided in addition to at least one camera, the at least one laser sensor is further adapted to detect a distance between the at least one reference surface and the patient transfer apparatus. The at least one camera is configured to adjust their settings according to input acquired from the at least one laser sensor, so as to maintain the at least one encoding index of the at least one tracking feature in sufficient focus. In a further embodiment, the acquisition of the inputs of both the at least one laser sensor and the at least one camera are continuous, such that the at least one encoding index of the at least one tracking feature is continuously kept within an ideal focal distance within the camera's field of view for optimal data interpretation. In an alternate embodiment, a first encoding index of the at least one tracking feature is adapted for resolving the gross position of the patient transfer apparatus within the at least one reference surface, and a second encoding index of the at least one tracking feature is adapted for resolving the fine position of the patient transfer apparatus within the at least one reference surface. The at least one camera is adapted to detect the first encoding index, and the at least one laser sensor is adapted to detect the second encoding index. An example of such a tracking feature may be a shape, point or series of points, line or series of lines, or some combination thereof etched into the reference surface with variable depth or reflectivity inbuilt, and wherein shape, point or series of points, line or series of lines, or some combination thereof changes according to segments of the reference surface and the depth or reflectivity varies at particular points within the segment of the reference surface.

Alternately or in addition to the previous embodiments, the patient transfer apparatus may be provided with one or more additional sensors capable of providing additional information related to the position or orientation of the patient transfer apparatus not obtainable or derivable from the information provided by other sensors. For example, rather than utilizing the input from multiple cameras or laser sensors oriented to detect the lateral, longitudinal, and vertical translational positions to mathematically derive or calculate the pitch, roll, or yaw of the patient transfer apparatus, at least one gyroscope may be provided to provide information regarding the rotational degrees of freedom of the patient transfer apparatus, while the at least one camera or the at least one laser sensor detect parameters related to the translational degrees of freedom. Additionally, an accelerometer may be provided to acquire input related to at least one of position, velocity, acceleration, jerk, and impulse. In an embodiment, accelerometer may be utilized to detect sudden instantaneous acceleration, such as when a clinician inadvertently bumps the patient out of the desired position, and may further be adapted to trigger an alert to the clinician when such an event happens. All these inputs as described in the preceding embodiments may be further supplemented by other inputs, such as external tracking units.

Once the at least one sensor has acquired input related to the position and orientation of the patient transfer apparatus, the system is further configured to interpret and provide information to inform patient positioning and treatment to the clinician or to the imaging or treatment modality. To accomplish this, the system is further provided with at least one processor, configured to process and convert the input from the at least one sensor to human or machine usable information such as coordinates and angles of rotation, among other derived statistics related to the position or orientation of the patient transfer apparatus, such as instantaneous or continuous movement, acceleration, or rotational degrees of freedom such as pitch, roll, or yaw. Additionally, the system is provided with at least one transmitter configured to transmit the input from the at least one sensor to the at least one processor. The at least one sensor is connected to the at least one transmitter which is in turn connected to the at least one processor, such that the at least one transmitter is capable of transmitting the input from the at least one sensor to the at least one processor for processing and conversion into coordinates within a reference from in at least one degree of freedom. The transmitter may transmit the input from the at least one sensor via wireless means, including but not limited to WiFi or Bluetooth®, or through physical means such as physical wire or other conduit. The at least one processor may similarly be connected to the at least one transmitter by wireless means or physical means. The at least one processor may be disposed in a separate unit such as a computer or mobile device or in an existing terminal or other output of the imaging or treatment modality, or it may be contained within the patient transfer apparatus. The system may additionally be provided with at least one power source for the at least one sensor, the at least one transmitter, and any additional powered components which may be disposed within the system, especially within the patient transfer apparatus. The at least one power source may include one or more of the following non-limiting options: batteries (more preferably MR compatible batteries, and even more preferably exchangeable or rechargeable in addition to being MR compatible), wired power supplies, and inductive power systems. In a further preferred embodiment, the imaging or treatment modality may provide a means of energizing the at least one power source. In yet a further preferred embodiment, the imaging modality is an MR scanner and the at least one power source is capable of using the electromagnetic field emitted by the MR scanner to energize the at least one power source.

With all of these components, the system is capable of taking the input from the at least one sensor as transmitted to it by the at least one transmitter and interpreting it into coordinate information within the reference frame corresponding to unique position and orientation information for the patient transfer apparatus. According to another aspect of this invention, a method of determining the coordinates of a patient transfer apparatus within a reference frame is provided. The method has the following steps:
a. Positioning an at least one sensor coupled to patient transfer apparatus such that it is capable of detecting at least one tracking feature positioned within a reference surface
b. Activating the at least one sensor such that it is capable of acquiring input including at least one tracking feature disposed in the reference surface and representing a unique position defined in the reference
c. Acquiring, using the at least one sensor, input which includes at least one tracking feature disposed in the reference surface, the at least one tracking feature representing a unique position within the reference surface
d. Transmitting, using the at least one transmitter, the input from the at least one sensor to at least one processor
e. Interpreting, using the at least one processor, the input from the at least one sensor, including translating selected structures of the at least one tracking feature into coordinates within the reference frame

In a further embodiment, the method further comprises one or more of the following additional steps outlined below.
f. Transmitting an output including the coordinates within the reference frame to another unit, such as a unit comprising a display. or
f. Transmitting an output including the coordinates within the reference frame at the rotation of the patient support apparatus within the reference frame to another unit, such as a unit comprising a display.

In a yet further embodiment, step e of the method outlined above may further have the following substeps:
e1. Detecting at least one encoding index disposed within the at least one tracking feature
e2. Identifying a structure identifier based the at least one encoding index
e3. Converting the structure identifier to a set of coordinates within the reference frame
e4. Outputting the appropriate coordinates within the reference frame as determined in one or more of the preceding substeps.
e5. Acquiring the coordinates within the reference frame of plural sensors according to the preceding steps
e6. Calculating an angle of rotation of the patient transfer apparatus based on the coordinates within the reference frame of the plural sensors.
e7. Outputting the angle of rotation of the patient transfer as calculated in step e6

In a yet further embodiment, the method may further have the following steps between steps e1 and e2:
e1.1. Manipulating the input obtained to make it suitable for interpretation.
e1.2. Detecting an encoding index disposed within the at least one tracking feature configured to provide information about a rotation of the patient transfer apparatus about an axis within the reference frame
e1.3. Determining an angle of the patient transfer apparatus within the reference frame

In accordance with another aspect of this invention, a method of using a system configured to support a patient and track the coordinates of a patient transfer apparatus within a reference frame. The method has the following steps:
a. Positioning a patient transfer apparatus on at least one of a support surface or an imaging or treatment modality, wherein at least one of the support surface or imaging or treatment modality further has at least one reference surface,
b. Positioning a patient on the patient transfer apparatus,
c. Moving the patient transfer apparatus,
d. Acquiring the new coordinates of the patient transfer apparatus

In a further embodiment, the method may include the following step between steps b and c:
b1. Acquiring the initial coordinates of the patient transfer apparatus within a reference frame,

In a further embodiment, step c of the above described method may further have the following substeps:
c1. Activating at least one air source connected to the patient transfer apparatus
c2. Converting at least one air bearing from a first deflated state to a second inflated state, thereby creating a region of reduced friction underneath the patient transfer apparatus,
c3. Moving the patient transfer apparatus,
c4. Deactivating the at least one air source connected to the patient transfer apparatus

In yet a further embodiment, the acquisition of the coordinates of the patient transfer apparatus within a reference frame may be continuous, including while the patient transfer apparatus is moving.

Additional aspects of the invention are further outlined below:

### Short Summary

This invention provides a method for tracking a transfer device based on an embedded optical sensor for determining position and orientation based on encoding structures. The functional components for this method are implemented in the form of an optical sensor within a tracking module for raw data acquisition, a surface with position-coded structures recognized by the tracking module, a transmission unit for data transmission, an evaluation unit including an algorithm for decoding and communicating the resulting information. Intended Benefit

The method was developed to enable precise and robust tracking of a free moving transfer device. It does not restrict the workflow of the clinical team or requires individual intermediate steps for tracking and thereby saves time. The clinical team will have continuous tracking information for use in medical imaging or treatment.

### Implementation and Invention Features

A tracking module is designed in such a way that it can be attached to or installed into a variety of transfer systems and or devices. At least one tracking module is used to extract information about multiple degrees of freedom of the position and orientation of said transfer system. In a preferred embodiment a multitude of tracking modules are used to achieve higher robustness, create redundancy and/or enhance the tracking accuracy.

The actual placement of the tracking module on the tracked system is designed to ensure that tracking is possible at all times. Placements closer to the center of a transfer device may be more favorable as they are less prone from overhanging the table.

The sensors inside the tracking module are used to record optical signals, e.g. in the form of images, preferably directed towards underneath the transfer device. This is realized with the use of embedded cameras, which acquire images of structured surface. Inside a single tracking module at least one optical sensor/camera is mounted.

An inbuilt light source serves to illuminate the structured surface to increase the independence from external conditions. In a preferred embodiment the light source consists of LEDs. The power source can be embodied by batteries, preferably MR-compatible ones. A battery pack may be implemented exchangeable or rechargeable. Alternative variants may consist of a wired power supply.

The structured surface is preferably underneath the transfer system. Further implementations may contain structured surfaces on angled surfaces with respect to the transfer device subsurface. These can benefit from angled tracking modules with cameras pointing towards these angled surfaces. The structured surface is produced either directly as a printed sheet or can be applied indirectly by fixing a printed film to a surface. The structured surface can consist of more than one continuous section to selectively cover specific locations.

On the structured surface, there are a set of preferably unique structures which differ in their features across the spatial area of search (area for the degrees of freedom in position and orientation). Size and relative arrangement of the structures is defined together with the tracking module in such a way that the module can always view and decode a structure. In the current implementation, this causes slightly more than 4 structures arranged in a square to fall within a field of view of the camera. Additional space between structure arrangement and the edge of the field of view is necessary to avoid cutting off structures during rotation and to be able to view a complete structure even in this case. This causes the maximum size of a structure to be related directly to the distance from the camera to the structured surface, as well as the camera viewing angle.

The features of the structures may contain binary coded information to discretely span the space of search. The feature expressions in binary coding, may be implemented by the presence or absence of geometric shapes (e.g., circles) at predefined positions within each structure. Alternative versions of structures featuring analog coding can also be implemented by calculating information about e.g. lengths and widths of specific geometric features from the image data. The orientation of straight lines in the acquired image, in relation to the reference, can be used as a feature expression for the calculation for the orientation of the transfer device. In a preferred embodiment information of 3 degrees of freedom are encoded by the feature expressions. These consist of 2 lateral and one rotational degree on an even plane of the patient table. Alternative variants may comprise a different number of degrees of freedom.

The transmission unit is preferably installed inside the tracking module and ensures the transmission of the recorded raw data. It includes software for communication with at least one camera and ensures its operability. As an on-board unit, a certain energy consumption is necessary and minimum space requirement is desired. The transmission is preferably carried out wirelessly by e.g. WiFi or Bluetooth® connection. Other variants can utilize wired interfaces (e.g. electric or opto-electric).

The evaluation unit is preferably located outside the tracking module to be less constrained in computing resources, energy consumption and physical space. Alternative variants may utilize integration of the evaluation unit into the on-board tracking module. The evaluation unit handles the processing of the raw data using the decoding algorithm, as well as the communication of the resulting information about the degrees of freedom.

The communication of position and orientation can be visualized to the user or can initiate actuating functions, such as slice repositioning in a medical imaging system, via appropriate interfaces. Active tracking could be triggered or inhibited by an external signal. It is possible to implement this via a switch or a wireless signal. This signal may be triggered by the user or by another system such as the scanner.

The decoding algorithm includes aspects of (conventional) image processing methods (current implementation). This comprises the use of proper image filters and multiple Hough transformations. Parts of the algorithm may be implemented by artificial intelligence methods, as well as model-based methods by integrating the user's workflow as a priori information, for example in the form of movement characteristics.

Position decoding preferably takes place in a stationary state, with a fixed focused camera. Decoding during motion based on an air bearing would require further implementations, such as additional cameras, mechanical distance fixations of the camera to the background, image corrections in the evaluation unit, auto-focus.

### Algorithm Description

Read-in of camera raw data.
Rotation of the image according to the structures'edge orientation
   Information of orientation (angle)
Crop image to only one valid full structure
   Optional information: Offset position in field of view
Head-/Feet-first marker detection (second straight line) and conditional image flip
   Flag the orientation
Circle detection inside chosen structure
   Knowledge about all circle positions
Create structure ID based on circle positions
   Binary code identifying the individual structure
Convert structure ID to x|y coordinates
   Information of position
Communication/visualization of position and orientation

Further additional aspects of the invention are additionally listed here:
Aspect 1. A system configured to support a patient and track the position and/or orientation of a patient support, the system comprising:
   A patient transfer apparatus configured to support and transfer a body of
   a patient;
   At least one reference surface:
   At least one sensor capable of acquiring input related to the position of the patient transfer apparatus;
   At least one processor, configured to process and convert the input from the at least one sensor;
   At least one transmitter, configured to transmit the input from the at least one sensor to the at least one processor;
   And
   At least one tracking feature disposed on the reference surface, having at least one encoding index adapted to be detected by the at least one sensor, and further representing a unique position within the reference surface;
   Wherein the at least one sensor is coupled to the patient transfer apparatus such that the at least one sensor is positioned to detect the at least one tracking feature disposed in a reference surface, and
   wherein the at least one sensor is connected to the at least one transmitter, and further wherein the at least one transmitter is connected to the at least one processor such that the at least one transmitter is capable of transmitting the input from the at least one sensor to the at least one processor for processing and conversion into coordinates within a reference frame in at least one degree of freedom.
Aspect 2. The system of aspect 1, wherein the system further comprises a support surface configured to support the patient transfer apparatus.
Aspect 3. The system of aspect 2, wherein the system further comprises an imaging or treatment modality, and further wherein the reference surface is incorporated into at least one of the support surface or the structure of the imaging or treatment modality.
Aspect 4. The system of aspect 3, wherein the support surface is integrated into the imaging or treatment modality.
Aspect 5. The system of aspect 3, wherein the system is capable of determining the position and orientation of the patient transfer apparatus in at least three degrees of freedom.
Aspect 6. The system of aspect 5, wherein the system is capable of determining the position and orientation of the patient transfer apparatus in at least six degrees of freedom.
Aspect 7. The system of aspect 3, wherein plural tracking features are disposed on at least the support surface, and further wherein the support surface defines reference surface, and wherein the at least one sensor is coupled to an underside of the patient transfer apparatus.
Aspect 8. The system of aspect 1, having plural sensors, and further wherein the plural sensors are selected from at least one of a camera, a charge coupled device, an infrared sensor, a laser sensor, a LIDAR sensor, a gyroscope, an accelerometer, or a haptic sensor.
Aspect 9. The system of aspect 8, wherein the at least one of the plural sensors is one or more cameras, and further wherein at least one sensor is provided with at least one means of illumination of the field of view of the camera, such that at least one encoding index of the at least one tracking feature is visible in the field of view of the one or more cameras and images generated by the one or more cameras are able to be interpreted by the at least one processor.
Aspect 10. The system of aspect 9, wherein the plural sensors are at least one camera and at least one laser sensor.
Aspect 11. The system of aspect 10, wherein the at least one camera is capable of detecting an at least one encoding index of the at least one tracking feature adapted for gross position within the reference surface, and further wherein the at least one laser sensor is capable of detecting another at least one encoding index of the at least one tracking feature adapted for fine position within the reference surface.
Aspect 12. The system of aspect 10, wherein the at least one laser sensor is capable of detecting a distance between the reference surface and the patient transfer apparatus, and further wherein the at least one camera is configured to adjust its settings to compensate for differences in distance between the reference surface and the patient transfer apparatus according to the input acquired from the at least one laser sensor, so as to maintain the at least one encoding index of the at least one tracking feature in sufficient focus such that the images generated by the at least one camera are able to be interpreted by the at least one processor.
Aspect 13. The system of aspect 8, wherein at least one of the plural sensors is at least one laser sensor configured to acquire corresponding changes in depth or reflectivity of the at least one tracking feature configured to represent the unique position of the at least one tracking feature that are able to be interpreted by the at least one processor.
Aspect 14. The system of aspect 1, further comprising a supplementary, external means of tracking position of the patient transfer device.
Aspect 15. The system of aspect 1, further comprising at least one air source and at least one air bearing located on an underside of the patient transfer apparatus, wherein the at least one air source, when activated, is configured to create airflow through the at least one air bearing such that a low-friction region is created underneath the patient transfer apparatus, thereby enabling reduced effort required to move the patient transfer apparatus.
Aspect 16. The system of aspect 1, wherein plural sensors are provided.
Aspect 17. The system of aspect 16, wherein the plural sensors are disposed on at least one of an inferior end, a superior end, or an intermediate region of the patient transfer apparatus.
Aspect 18. The system of aspect 17, wherein a first series of plural sensors is disposed at a superior end of the patient transfer apparatus and a second series of plural sensors is disposed at an inferior end.
Aspect 19. The system of aspect 18, wherein the first series of plural sensors is connected to a first at least one transmitter and the second series of plural sensors is connected to a second at least one transmitter, and wherein both the first and second at least one transmitter are connected to the at least one processor.
Aspect 20. The system of aspect 1, wherein the patient transfer apparatus is shaped such that it is capable of being rotated about an axis extending perpendicularly from a top side of the patient transfer apparatus.
Aspect 21. The system of aspect 1, wherein the at least one processor is physically coupled to the patient transfer apparatus.
Aspect 22. A method of using a system configured to support a patient and track the coordinates of a patient transfer apparatus within a reference frame, the method comprising the following steps:
   (a) Positioning a patient transfer apparatus on at least one of a support surface or an imaging or treatment modality, wherein at least one of the support surface or imaging or treatment modality further has at least one reference surface,
   (b) Positioning a patient on the patient transfer apparatus,
   (c) Moving the patient transfer apparatus,
   (d) Acquiring the coordinates of the patient transfer apparatus
Aspect 23. The method of aspect 22, wherein the method further comprises the following step inserted between steps b and c:
   b1. Acquiring the initial coordinates of the patient transfer apparatus within a
Aspect 24. The method of aspect 22, wherein step c is accomplished by the following substeps:
   c1. Activating at least one air source connected to the patient transfer apparatus
   c2. Converting at least one air bearing from a first deflated state to a second inflated state, thereby creating a region of reduced friction underneath the patient transfer apparatus,
   c3. Moving the patient transfer apparatus,
   c4. Deactivating the at least one air source connected to the patient transfer apparatus
Aspect 25. The method of aspect 22 or 23, wherein acquisition of the coordinates of the patient transfer apparatus within a reference frame is continuous, including while the patient transfer apparatus is moving.
Aspect 26. A method of determining the coordinates of a patient transfer apparatus within a reference frame, the method comprising the following steps:
   a. Positioning an at least one sensor coupled to patient transfer apparatus such that it is capable of detecting at least one tracking feature positioned within a reference surface
   b. Activating the at least one sensor such that it is capable of acquiring input including at least one tracking feature disposed in the reference surface and representing a unique position defined in the reference surface
   c. Acquiring, using the at least one sensor, input which includes at least one tracking feature disposed in the reference surface, the at least one tracking feature representing a unique position within the reference surface
   d. Transmitting, using the at least one transmitter, the input from the at least one sensor to at least one processor
   e. Interpreting, using the at least one processor, the input from the at least one sensor, including translating selected structures of the at least one tracking feature into coordinates within the reference frame.
Aspect 27. The method of aspect 26, further comprising the following step
   f. Transmitting an output including the coordinates within the reference frame to another unit, such as a display.
Aspect 28. The method of aspect 26, wherein step e of the method further comprises the following substeps:
   e1. Detecting at least one encoding index disposed within the at least one tracking feature
   e2. Identifying a structure identifier based on the at least one encoding index
   e3. Converting the structure identifier to a set of coordinates within the reference frame
   e4. Outputting the appropriate coordinates within the reference frame as determined in the preceding substeps.
Aspect 29. The method of aspect 28, wherein the method further comprises the following step between steps e1 and e2:
   e1.1. Manipulating the input obtained to make it suitable for interpretation.
Aspect 30. The method of aspect 28, wherein the method further comprises the following steps between steps e1 and e2:
   e1.2. Detecting an encoding index disposed within the at least one tracking feature configured to provide information about a rotation of the patient transfer apparatus about an axis within the reference frame
   e1.3. Determining an angle of the patient transfer apparatus within the reference frame
Aspect 31. The method of aspect 28, wherein the method further comprises the following steps:
   e5. Acquiring the coordinates within the reference frame of plural sensors according to the preceding steps
   e6. Calculating an angle of rotation of the patient transfer apparatus based on the coordinates within the reference frame of the plural sensors.
   e7. Outputting the angle of rotation of the patient transfer as calculated in step e6
Aspect 32. The method of one of aspect 30 or 31, further comprising the following step:
   f. Transmitting an output including the coordinates within the reference frame at the rotation of the patient support apparatus within the reference frame to another unit, such as a display.

1. A system configured to support a patient and track the position and/or orientation of a patient support, the system comprising:
a patient transfer apparatus configured to support and transfer a body of the patient;
at least one reference surface;
a sensor capable of acquiring input related to the position of the patient transfer apparatus;
at least one processor configured to process and convert the input from the at least one sensor;
at least one transmitter configured to transmit the input from the at least one sensor to the at least one processor;
a tracking feature disposed on the reference surface and having at least one encoding index adapted to be detected by the at least one sensor, and further representing a unique position within the reference surface; and
wherein the sensor is coupled to the patient transfer apparatus, such that the sensor is positioned to detect the tracking feature disposed in or on the at least one reference surface, and
wherein the sensor is connected to the at least one transmitter, and
wherein the at least one transmitter is connected to the at least one processor, such that the at least one transmitter is capable of transmitting the input from the sensor to the at least one processor for processing and conversion into coordinates within a reference frame in at least one degree of freedom.

2. The system of claim 1, wherein the system further comprises a support surface configured to support the patient transfer apparatus.

3. The system of claim 2, wherein the system further comprises an imaging or treatment modality, and wherein the at least one reference surface is incorporated into at least one of the support surface and a structure of the imaging or treatment modality.

4. (canceled)

5. The system of claim 3, wherein the system is capable of determining the position and orientation of the patient transfer apparatus in at least three degrees of freedom.

6. (canceled)

7. The system of claim 3, wherein plural tracking features are disposed on at least the support surface, and the support surface defines the at least one reference surface, and the at least one sensor is coupled to an underside of the patient transfer apparatus.

8. The system of claim 1, further having plural sensors being selected from at least one of a camera, a charge coupled device, an infrared sensor, a laser sensor, a LiDAR sensor, a gyroscope, an accelerometer, and a haptic sensor.

9. The system of claim 8, wherein the plural sensors comprises at least one camera, and the at least one camera is provided with at least one means of illumination of the field of view of the at least one camera, such that at least one encoding index of the at least one tracking feature is visible in the field of view of the at least one camera and images generated by the at least one camera are able to be interpreted by the at least one processor.

10. (canceled)

11. The system of claim 9, wherein the at least one camera is capable of detecting one of the at least one encoding index of the at least one tracking feature, the one of the at least one encoding index being adapted for gross position within the at least one reference surface, and the at least one laser sensor is capable of detecting another of the at least one encoding index of the at least one tracking feature, the another of the at least one encoding index being adapted for fine position within the at least one reference surface.

12. The system of claim 9, wherein the at least one laser sensor being capable of detecting a distance between the at least one reference surface and the patient transfer apparatus, and the at least one camera is configured to adjust its settings to compensate for differences in distance between the at least one reference surface and the patient transfer apparatus according to the input acquired from the at least one laser sensor, so as to maintain the at least one encoding index of the at least one tracking feature in sufficient focus such that the images generated by the at least one camera are able to be interpreted by the at least one processor.

13. The system of claim 8, wherein the plural sensors comprises at least one laser sensor configured to acquire corresponding changes in depth or reflectivity of the at least one tracking feature so as to represent the unique position of the at least one tracking feature, which can be interpreted by the at least one processor.

14. The system of claim 1, further comprising a supplementary, external means of tracking position of the patient transfer apparatus.

15. The system of claim 1, further comprising at least one air source and at least one air bearing located on an underside of the patient transfer apparatus, wherein the at least one air source, when activated, is configured to create airflow through the at least one air bearing such that a low-friction region is created underneath the patient transfer apparatus, thereby enabling reduced effort required to move the patient transfer apparatus.

16. (canceled)

17. (canceled)

18. (canceled)

19. (canceled)

20. The system of claim 1, wherein the patient transfer apparatus is shaped such that it is capable of being rotated about an axis extending perpendicularly from a top side of the patient transfer apparatus.

21. The system of claim 1, wherein the at least one processor is physically coupled to the patient transfer apparatus.

22. A method of using a system configured to support a patient and track the coordinates of a patient transfer apparatus within a reference frame, the method comprising the following steps:
(a) positioning the patient transfer apparatus configured to support the patient on at least one of a support surface and an imaging or treatment modality, wherein at least one of the support surface and imaging or treatment modality has at least one respective reference surface, (b) moving the patient transfer apparatus, and (c) acquiring the new coordinates of the patient transfer apparatus.

23. The method of claim 22, wherein the method further comprises:
(b1) acquiring the initial coordinates of the patient transfer apparatus within a reference frame; and
wherein step (b1) occurs between steps (b) and (c).

24. The method of claim 22, wherein step (c) comprises:
(c1) activating at least one air source connected to the patient transfer apparatus,
(c2) converting at least one air bearing from a first deflated state to a second inflated state, thereby creating a region of reduced friction underneath the patient transfer apparatus,
(c3) moving the patient transfer apparatus, and
(c4) deactivating the at least one air source connected to the patient transfer apparatus.

25. (canceled)

26. A method of determining the coordinates of a patient transfer apparatus within a reference frame, the method comprising the following steps:
(a) positioning at least one sensor coupled to the patient transfer apparatus, such that the at least one sensor is capable of detecting at least one tracking feature positioned within the at least one reference surface;
(b) activating the at least one sensor, such that it is capable of acquiring input, the input including the at least one tracking feature disposed in the at least one reference surface and the at least one tracking feature represents a unique position defined in the at least one reference surface;
(c) acquiring, using the at least one sensor, input, the input including the at least one tracking feature disposed in the at least one reference surface, the at least one tracking feature representing a unique position within the at least one reference surface;
(d) transmitting, using the at least one transmitter, the input from the at least one sensor to at least one processor;
(e) interpreting, using the at least one processor, the input from the at least one sensor, including converting selected structures of the at least one tracking feature into coordinates within the reference frame; and (f) transmitting output, the output including coordinates within the reference frame to another unit comprising a display.

27. (canceled)

28. The method of claim 26, wherein step (e) further comprises:
(e1) detecting at least one encoding index disposed within the at least one tracking feature;
(e2) identifying a structure identifier based the at least one encoding index;
(e3) converting the structure identifier to a set of coordinates within the reference frame;
(e4) outputting the appropriate coordinates within the reference frame.

29. The method of claim 28, further comprising:
(e1.1) manipulating the input obtained to make it suitable for interpretation;
and wherein step (e1.1) occurs between steps (e1) and (e2).

30. The method of claim 28, further comprising:
(e1.1) detecting an encoding index disposed within the at least one tracking feature, the encoding index configured to provide information about a rotation of the patient transfer apparatus about an axis within the reference frame;
(e1.2) determining an angle of the patient transfer apparatus within the reference frame; and
wherein steps (e1.1) and (e1.2) occur between steps (e1) and (e2).

31. The method of claim 28, further comprising:
(e5) acquiring the coordinates within the reference frame of the at least one sensor;
(e6) calculating an angle of rotation of the patient transfer apparatus based on the coordinates within the reference frame of the at least one sensor; and
(e7) outputting the angle of rotation of the patient transfer as calculated in step (e6).

32. The method of claim 30, further comprising:
(f) transmitting an output, the output including the coordinates within the reference frame at the rotation of the patient support apparatus within the reference frame to another unit comprising a display.
